(19) United States
(12) Patent Application Publication
(10) Pub. No.: US 2019/0367436 A1
(43) Pub. Date: Dec. 5, 2019

(54) IMPROVED PROCESS OF MAKING BIODERIVED PROPYLENE GLYCOL

(71) Applicant: Archer Daniels Midland Company, Decatur, IL (US)

(72) Inventor: Chi Cheng Ma, Forsyth, IL (US)

(21) Appl. No.: 16/477,511

(22) PCT Filed: Jan. 10, 2018
(86) PCT No.: PCT/US18/13112

§ 371 Date: Jul. 11, 2019

(51) Int. Cl. C07C 29/60

(52) U.S. Cl. CPC C07C 29/60

(57) ABSTRACT

An improved process for making bioderived propylene glycol by reacting a feed composition including glycerol with hydrogen in the presence of a catalyst, wherein the improvement involves causing the reaction to take place in the presence of one or more additives selected 10 from the group consisting of the soluble acetate, citrate, lactate, gluconate, propionate and glycerate salts.

### TECHNICAL FIELD

This invention relates generally to processes for making a bioderived propylene glycol (meaning, 1,2-propanediol).

### BACKGROUND ART

Commercial production of petroleum-based or -derived propylene glycol involves the hydration of propylene oxide, made predominantly by the oxidation of propylene. The commercial production of ethylene glycol similarly involves the hydration of ethylene oxide, made by the oxidation of ethylene. Propylene and ethylene in turn are industrial by-products of gasoline manufacture, for example, as by-products of fluid cracking of gas oils or steam cracking of hydrocarbons.

The world's supply of petroleum is, however, being depleted at an increasing rate. As the available supply of petroleum decreases or as the costs of acquiring and processing the petroleum increase, the manufacture of various chemical products derived therefrom (such as propylene glycol and ethylene glycol) will be made more difficult. Accordingly, in recent years much research has taken place to develop suitable biobased propylene glycol and ethylene glycol products, which can be interchangeable with propylene glycol and ethylene glycol products deriving from petroleum refining and processing methods but which are made from renewable versus nonrenewable materials.

As a result of these efforts, processes have been developed by several parties involving the hydrogenolysis of especially five and six carbon sugars and/or sugar alcohols, whereby the higher carbohydrates are broken into fragments of lower molecular weight to form compounds which belong to the glycol or polyol family. Sugars containing five carbon chains, such as ribose, arabinose, xylose and lyxose, lactic acid and five carbon chain sugar alcohols such as xylitol and arabinitol, are among the materials contemplated in U.S. Pat. No. 7,038,094 to Werpy et al., for example, while six carbon sugars such as glucose, galactose, maltose, lactose, sucrose, allose, altrose, mannose, gulose, idose and talose and six carbon chain sugar alcohols such as sorbitol are addressed by U.S. Pat. No. 6,841,085 to Werpy et al. (hereafter, “Werpy '085”). Some of these carbohydrate-based feedstocks are commercially available as pure or purified materials. These materials may also be obtained as side-products or even waste products from other processes, such as corn processing. The sugar alcohols may also be intermediate products produced in the initial stage of hydrogenating a sugar.

For other known examples of such processes, U.S. Pat. No. 5,206,927 describes a homogeneous process for hydrocracking carbohydrates in the presence of a soluble transition metal catalyst to produce lower polyhydric alcohols. A carbohydrate is contacted with hydrogen in the presence of a soluble transition metal catalyst and a strong base at a temperature of from about 25° C. to about 200° C. and a pressure of from about 15 to about 3000 psi. However, as is evident from Tables II and III in the disclosure of U.S. Pat. No. 5,206,927, about 2-7% of other polyol compounds are produced in the hydrocracking process. U.S. Pat. No. 4,476,331 describes a two stage method of hydrocracking carbohydrates using a modified ruthenium catalyst. European Patent Applications EP-A-0523 014 and EP-A-0 415 202 describe a process for preparing lower polyhydric alcohols by catalytic hydrocracking of aqueous sucrose solutions at elevated temperature and pressure using a catalyst whose active material comprises the metals cobalt, copper and manganese. Still other examples of such carbohydrate-based processes may be found without difficulty by those skilled in the art.

Other efforts have been based on the use of another readily accessible biobased feedstock, namely, glycerol. Glycerol is currently produced as a byproduct in making biodiesel from vegetable and plant oils, through the transesterification reaction of lower alkanols with higher fatty acid triglycerides to yield lower alkyl esters of higher fatty acids and a substantial glycerol byproduct. Glycerol is also available as a by-product of the hydrolysis reaction of water with higher fatty acid triglycerides to yield soap and glycerol. The higher fatty acid triglycerides may derive from animal or vegetable (plant) sources, or from a combination of animal and vegetable sources as well known, and a variety of processes have been described or are known.

A biobased glycerol is also available as a product of the hydrogenolysis of sorbitol, as described in an exemplary process in U.S. Pat. No. 4,366,332, issued Dec. 28, 1982.

U.S. Pat. Nos. 5,276,181 and 5,214,219 describe a process of hydrogenolysis of glycerol using copper and zinc catalyst in addition to sulfided ruthenium catalyst at a pressure over 2100 psi and temperature between 240-270° C.

U.S. Pat. No. 5,616,817 describes a process of preparing 1,2-propanediol (more commonly, propylene glycol) by catalytic hydrogenolysis of glycerol at elevated temperature and pressure using a catalyst comprising the metals cobalt, copper, manganese and molybdenum.

German Patent DE 541362 describes the hydrogenolysis of glycerol with a nickel catalyst.

Persoa & Tundo (Ind. Eng. Chem. Res. 2005, 8535-8537) describe a process for converting glycerol to 1,2-propanediol by heating under low hydrogen pressure in presence of Raney nickel and a liquid phosphonium salt. Selectivities toward 1,2-propanediol as high as 93% were reported, but required using a pure glycerol and long reaction times (20 hrs).

Crabtree et al. (Hydrocarbon processing February 2006 pp 87-92) describe a phosphine/precious metal salt catalyst that permit a homogenous catalyst system for converting glycerol into 1,2-propanediol. However, low selectivity (20-30%) was reported.

Other reports indicate use of Raney copper (Montassier et al. Bull. Soc. Chim. Fr. 2 1989 148; Stud. Surf. Sci. Catal. 41 1988 165), copper on carbon (Montassier et al. J. Appl. Catal. A 121 1995 231)), copper-platinum and copper ruthenium (Montassier et al. J. Mol. Catal. 70 1991 65). U.S. Pat. No. 7,790,937 to Henkelmann et al. similarly describes converting a glycerol-containing stream, especially a glycerol-containing stream obtained from biodiesel production, to propylene glycol by reaction with hydrogen in the presence of a heterogeneous catalyst containing copper. Raney copper and copper-containing metal alloys in the form of a Raney catalyst are mentioned as preferred.

Still other homogenous catalyst systems such as tungsten and Group VIII metal-containing catalyst compositions have also been tried (U.S. Pat. No. 4,642,394). Miyazawa et al. (J. Catal. 240 2006 213-221) & Kusunoki et al (Catal. Comm. 6 2005 645-649) describe a Ru/C and ion exchange resin for conversion of glycerol in aqueous solution.

The previously-cited Werpy '085 reference contemplates conversion of a composition including glycerol to bioderived propylene glycol by reaction with hydrogen in the presence of a Re-containing multimetallic catalyst.

Numerous other examples of like processes may be found without difficulty by those skilled in the art.

One of the recognized problems in producing a biobased propylene glycol or ethylene glycol by any of these methods, however, is that other diol compounds are formed (e.g., four carbon and higher diols) to varying degrees in all of these processes. The boiling points of many of these materials are very close to one another, so that the separation of high purity bioderived propylene glycol from these other polyhydric alcohols is exceedingly difficult by conventional distillation methods—such that substantial amounts of the desired propylene glycol product are inevitably co-distilled with the higher diols in order to remove these to the extent needed for many commercial applications, in addition to involving substantial expense.

Several reports in the literature describe efforts for azeotropically separating the other polyhydric alcohols from propylene glycol. For instance, U.S. Pat. No. 4,935,102 describes a method for using an azeotrope forming agent such as propylene glycol isobutyl ether, tetrahydrofurfuryl alcohol, N,N-dimethylacetamide, ethylene glycol diethyl ether, diethylene glycol diethyl ether, 2-methoxyethyl ether, ethylene glycol n-butyl ether, diacetone alcohol and ethyl n-butyl ketone. In U.S. Pat. No. 5,423,955, the azeotrope forming agent consists of a material selected from the group consisting of toluene, ethyl benzene, o-xylene, p-xylene, cumene, m-diisopropyl benzene, m-diethyl benzene, mesitylene, p-cymene, hexane, cyclohexane, methyl cyclohexane, heptane, 3-methyl pentane, octane, decane, 2,3,4-trimethyl pentane, dipentene, decalin, dicyclopentadiene, alpha-phellandrene, limonene, hemimellitene, myrcene, terpinolene, p-mentha-1,5-diene, beta-pinene, 3-carene, 1-heptene, cyclopentane, pentane, o-diethyl benzene, 2,2-dimethyl butane and 2-methyl butane.

Alternative approaches to purifying the product mixture have been proposed in U.S. Pat. No. 8,143,458 to Kalagias, wherein the addition of a polar solvent and extractive distillation are presented as an alternative to the use of an azeotropic agent, and in U.S. Pat. No. 8,177,980 to Hilaly et al., wherein simulated moving bed chromatography is offered as a means to achieve a purified, commercial grade biobased propylene glycol.

Nevertheless, the separation of a number of byproducts from the desired bioderived propylene glycol remains difficult and costly. The presence of four carbon and higher diols can in particular mean substantial losses of co-distilled propylene glycol product where conventional distillation methods are desired to be used for product purification, so that a process which enables a lesser amount of byproduct higher diols would be welcomed.

### SUMMARY OF THE INVENTION

The following presents a simplified summary of the invention in order to provide a basic understanding of some of its aspects. This summary is not an extensive overview of the invention and is intended neither to identify key or critical elements of the invention nor to delineate its scope. The sole purpose of this summary is to present some concepts of the invention in a simplified form as a prelude to the more detailed description that is presented later.

In one aspect, the present invention concerns a process for making bioderived propylene glycol by reacting a feed composition including glycerol with hydrogen in the presence of a suitable catalyst under conditions effective to carry out the conversion, wherein one or more additives selected from the group consisting of the soluble (in the glycerol-containing feed composition) acetate, citrate, lactate, gluconate, propionate and glycerate salts are combined with or included in the feed composition. We have found that the use of such additives can result in greater propylene glycol yields, as compared to processes for the hydrogenolysis of the same glycerol-containing feeds under the same conditions, but in the absence of the additives. Without being limiting of the present invention, it is believed that the additives of the present invention play a role in reducing the number and/or the extent of the various side reactions that commonly occur in all of the aforementioned known glycerol hydrogenolysis methods for producing 1,2-propanediol (or propylene glycol). In preferred embodiments, one or more acetate salts are employed, as the acetate salts can reduce the amount of four carbon and greater diols in addition to enabling greater propylene glycol yields.

In certain embodiments, the additives are used in a process of a type described in Patent Cooperation Treaty Application No. PCT/US17/60187, filed Nov. 6, 2017 for “Process for Producing 1,2-Propanediol from Glycerol”, wherein a biobased propylene glycol is produced from an essentially anhydrous glycerol-containing feed, especially an essentially anhydrous glycerol-containing feed obtained by removing water from a product mixture resulting from the hydrogenolysis of the glycerol-containing feed and by partially separating the desired propylene glycol product from unreacted glycerol, with recycling at least a part of the remaining combined propylene glycol and glycerol and combining this recycle with a refined glycerol product, especially a USP grade glycerol product, which typically is at least about 99.5 to 99.7% pure glycerol with a corresponding maximum moisture content of 0.5% to 0.3% by weight.

The resulting essentially anhydrous feed, where “essentially anhydrous” refers to a feed which contains less than 5 weight percent of water, preferably less than 3 weight percent, more preferably less than 2 weight percent, still more preferably less than 1 weight percent and even more preferably less than 0.5 weight percent of water, is thus comprised of a recycle component and a makeup, refined glycerol component. As a consequence, it will be particularly desirable in the context of such processes to reduce the number and/or the extent of side reactions that occur in the hydrogenolysis of the glycerol-containing feed by means of the present additives, and in particular to reduce the production of those byproducts which are separated from 1,2-propanediol only with great difficulty and/or expense (such as the four carbon and higher diols) and that might therefore pose a risk of being built up over time, except (in the absence of the use of the additives of the present invention) through the use of exceptional and expensive measures to separate these byproducts out and avoid their recycle.

### DETAILED DESCRIPTION OF EMBODIMENTS

As used in this application, the singular forms “a”, “an” and “the” include plural references unless the context clearly indicates otherwise. The term “comprising” and its derivatives, as used herein, are similarly intended to be open ended terms that specify the presence of the stated features, elements, components, groups, integers, and/or steps, but do not exclude the presence of other unstated features, elements, components, groups, integers and/or steps. This understanding also applies to words having similar meanings, such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers, and/or steps. The term “consisting essentially of”, as used herein, is intended to specify the presence of the stated features, elements, components, groups, integers, and/or steps, as well as those that do not materially affect the basic and novel characteristic(s) of stated features, elements, components, groups, integers, and/or steps.

Unless otherwise indicated, any definitions or embodiments described in this or in other sections are intended to be applicable to all embodiments and aspects of the subjects herein described for which they would be suitable according to the understanding of a person of ordinary skill in the art.

As indicated above, the present invention in one aspect relates to a process for making bioderived propylene glycol by reacting a feed composition including glycerol with hydrogen in the presence of a suitable catalyst under conditions effective to carry out the conversion, wherein one or more additives selected from the group consisting of the soluble (in the glycerol-containing feed composition) acetate, citrate, lactate, gluconate, propionate and glycerate salts are combined with or included in the feed composition.

In preferred embodiments, one or more acetate, glycerate or lactate salts are combined with or included in the feed composition in amounts sufficient to improve the yield of propylene glycol by at least fifteen (15) percent, preferably by at least twenty (20) percent, more preferably by at least thirty (30) percent and still more preferably by at least forty (40) percent over that achieved in the absence of the additives under the same conditions. The degree of improvement that can be realized can be expected to vary dependent on the particular hydrogenolysis method and catalyst used and upon the conditions under which a particular process is carried out, but in general it is expected that the additives of the present invention will be effective for improving at least to some degree the yield realized from any of the known hydrogenolysis catalysts and methods. For example, in the context of a process using a Re-containing multimetallic catalyst for the hydrogenolysis of glycerol as taught in Werpy '085, more specifically, a nickel/rhenium on carbon catalyst, 0.03 mols of the soluble lactate, glycerate and acetate salts were sufficient under the conditions specified in the examples below to provide yields 17.6 percent, 22.2 percent and 47.5 percent greater, respectively, than those realized with the same catalyst, under the same conditions but in the absence of the soluble lactate, glycerate and acetate salts.

In certain embodiments, the additives are used in a process of a type described in Patent Cooperation Treaty Application No. PCT/US17/60187, filed Nov. 6, 2017 for “Process for Producing 1,2-Propanediol from Glycerol”, wherein a biobased propylene glycol is produced from an essentially anhydrous glycerol-containing feed, especially an essentially anhydrous glycerol-containing feed obtained by removing water from a product mixture resulting from the hydrogenolysis of the glycerol-containing feed and by partially separating the desired propylene glycol product from unreacted glycerol, with recycling at least a part of the remaining combined propylene glycol and glycerol and combining this recycle with a refined glycerol product, especially a USP (United States Pharmacopeia) grade glycerol product, which typically is at least 99.5 to 99.7% pure glycerol with a corresponding maximum moisture content of 0.5% to 0.3% by weight.

The resulting essentially anhydrous feed, where “essentially anhydrous” refers to a feed which contains less than 5 weight percent of water, preferably less than 3 weight percent, more preferably less than 2 weight percent, still more preferably less than 1 weight percent and even more preferably less than 0.5 weight percent of water, is thus comprised of a recycle component and a makeup, refined glycerol component. As a consequence, it will be particularly desirable in the context of such processes to reduce the number and/or the extent of side reactions that occur in the hydrogenolysis of the glycerol-containing feed by means of the present additives, and in particular to reduce the production of those byproducts which are separated from 1,2-propanediol only with great difficulty and/or expense (such as the four carbon and higher diols) and that might therefore pose a risk of being built up over time, except (in the absence of the use of the additives of the present invention) through the use of exceptional and expensive measures to separate these byproducts out and avoid their recycle.

The present invention is further demonstrated by the non-limiting examples that follow:

### Examples 1-6

120 milliliters of a feed comprised of a) a solution of 30 weight percent of glycerol in water, b) 0.03 mols of salts of a selected organic acid, c) 1. 4 grams of sodium hydroxide and d) 5 grams of a purchased nickel-rhenium (5% Ni-1% Re on Norit ROX carbon) (wet) on carbon catalyst were placed in a 300 mL stainless steel high pressure autoclave reactor, and reacted with hydrogen at 9.7 MPa, gauge (1400 pounds per square inch, gauge). The reactor contents were stirred at 600 rpm for three hours at 215 degrees Celsius, and the reactor contents were then filtered for analysis. The percentages of ethylene glycol, combined butanediols (BDO), combined pentanediols (PDO) and overall PG yield were then determined, and are reported in Table 1 below:

TABLE 1

| | EG/PG | BDO/PG | PDO/PG | PG Yield |
|---|---|---|---|---|
| Control | 7.63 | 2.73 | 0.93 | 37.38 |
| Acetate | 7.65 | 1.97 | 0.74 | 55.74 |
| Citrate | 7.61 | 2.76 | 0.91 | 41.28 |
| Lactate | 8.07 | 3.14 | 1.26 | 44.44 |
| Gluconate | 7.85 | 3.31 | 1.14 | 41.33 |
| Propionate | 8.72 | 4.66 | 2.16 | 42.95 |
| Glycerate | 8.23 | 3.09 | 1.10 | 46.16 |

What is claimed is:

1. In a process for making bioderived propylene glycol by
reacting a feed composition including glycerol with hydrogen in the presence of a catalyst, the improvement comprising causing the reaction to take place in the presence of one or more additives selected from the group consisting of the soluble acetate, citrate, lactate, gluconate, propionate and glycerate salts.

2. The improved process of claim 1, wherein one or more soluble acetate salts are used as the one or more additives.

3. The improved process of claim 1, wherein the catalyst is a Re-containing multimetallic catalyst.

4. The improved process of claim 1, wherein a glycerol-containing feed composition contains less than 5 weight percent of water.

5. The improved process of claim 4, wherein the glycerol-containing feed composition contains less than 3 weight percent of water.

6. The improved process of claim 5, wherein the glycerol-containing feed composition contains less than 2 weight percent of water.

7. The improved process of claim 6, wherein the glycerol-containing feed composition contains less than 1 weight percent of water.

8. The improved process of claim 7, wherein the glycerol-containing feed composition contains less than 0.5 weight percent of water.

9. The improved process of any one of claims 4-8, wherein the glycerol-containing feed composition consists of a product recycle portion from the process and a makeup portion of a virgin glycerol feed.

10. The improved process of claim 9, wherein the glycerol-containing feed composition consists essentially of 1,2-propanediol produced by the hydrogenolysis reaction combined with glycerol.

11. The improved process of claim 10, wherein the glycerol-containing feed composition contains less than 1 percent by weight of anything other than 1,2-propanediol, glycerol and water.

12. The improved process of claim 11, wherein the glycerol-containing feed composition contains from 5 to 50 weight percent of glycerol.

13. The improved process of claim 12, wherein the glycerol-containing feed composition contains from 10 to 40 weight percent of glycerol.

14. The improved process of claim 13, wherein the glycerol-containing feed composition contains from 20 to 30 weight percent of glycerol.

15. The improved process of claim 9, wherein the virgin glycerol feed is a refined, USP grade glycerol product that is at least 99.5 percent glycerol and which contains less than 0.5 weight percent of water.

16. The improved process of claim 9, further comprising a dewatering step to provide a glycerol-containing feed composition of the requisite dryness, performed on one or more of the crude product mixture from the hydrogenolysis reaction, a glycerol-containing fraction of the crude product mixture, the virgin glycerol feed or a combination of two or more of these.

17. The improved process of claim 16, wherein the glycerol-containing feed composition is produced by steps comprising limiting the per-pass conversion of glycerol in the hydrogenolysis to less than full conversion, distilling off overhead substantially all of the water and lighter byproducts in the resultant product mixture in a first, water removal column, passing the bottoms from the first, water removal column to a product recovery column wherein a saleable propylene glycol product of at least 95 percent purity is recovered overhead and the bottoms is used as the product recycle portion from the hydrogenolysis process.

18. The improved process of claim 17, wherein the saleable propylene glycol product from the product recovery column is at least 99.5 percent pure glycerol.

19. A process for producing biobased 1,2-propanediol, comprising:
reacting a glycerol-containing feed composition containing less than 5 weight percent of water with hydrogen in the presence of a heterogeneous copper-containing catalyst and in the presence of one or more additives selected from the group consisting of the soluble acetate, citrate, lactate, gluconate, propionate and glycerate salts, to partially convert glycerol in the glycerol-containing feed composition to a crude reaction product mixture including 1,2-propanediol;
removing water from the crude reaction product mixture;
recovering a portion but not all of the 1,2-propanediol from the crude reaction product mixture; and
recycling the remainder of the 1,2-propanediol with unconverted glycerol and combining these with makeup glycerol to provide additional of the glycerol-containing feed composition.

20. The process of claim 19, wherein the heterogeneous copper-containing catalyst is a copper alloy-based sponge metal catalyst prepared from an alloy comprising copper and aluminum.

21. The process of claim 20, wherein the catalyst further comprises zinc as a promoter.

22. The process of any of claims 19-21, carried out at a temperature of less than 250 degrees Celsius.

23. The process of claim 22, carried out at a temperature of less than 230 degrees Celsius.

24. The process of claim 23, carried out at a temperature of less than 215 degrees Celsius.

25. The process of any of claims 22-24, wherein by means of removing water from the crude reaction product mixture and by controlling the relative amounts used to provide the additional glycerol-containing feed composition of the recycled 1,2-propanediol and unconverted glycerol on the one hand and of makeup glycerol on the other hand, the additional glycerol-containing feed composition contains less than 3 weight percent of water.

26. The process of claim 25, wherein the additional glycerol-containing feed composition contains less than 2 weight percent of water.

27. The process of claim 26, wherein the additional glycerol-containing feed composition contains less than 1 weight percent of water.

28. The process of claim 27, wherein the additional glycerol-containing feed composition contains less than 0.5 weight percent of water.
